(19) United States
(12) Patent Application Publication
(10) Pub. No.: US 2009/0247603 A1
(43) Pub. Date: Oct. 1, 2009

(54) Stabilized pharmaceutical compositions comprising an HMG-CoA reductase inhibitor

(75) Inventor: Laxminarayan Joshi, Toronto (CA)

Correspondence Address: PEACOCK MYERS, P.C., 201 THIRD STREET, N.W., SUITE 1340, ALBUQUERQUE, NM, 87102, US

(73) Assignee: Orbus Pharma, Inc., Markham (CA)

(21) Appl. No.: 11/315,986

(22) Filed: Dec. 23, 2005

(51) Int. Cl. A61K 31/40

(52) U.S. Cl. 514/423

(57) ABSTRACT

The present invention is a new stable drug composition particularly suitable for use as an antihypercholesterolaemic or antihyperlipidaemic agent. The present invention is specifically a drug composition comprising a pharmaceutical, a complexing agent and a surfactant, and a method for manufacturing same. When applied to unstable drugs with low solubility and poor bioavailability, like HMG-CoA reductase inhibitors and especially atorvastatin calcium amorphous form, the resulting drug composition is more stable and is characterized by an improved dissolution profile.

### FIELD OF THE INVENTION

The present invention is a new stable pharmaceutical composition suitable for use as an antihypercholesterolemic or antihyperlipidaemia agent, and more particularly a stable pharmaceutical composition containing as an active substance, a 3-hydroxy-3-methylglutaryl coenzyme A reductase (HMG-CoA reductase) inhibitor.

### BACKGROUND OF THE INVENTION

Statins, their derivatives, analogues and pharmaceutically acceptable salts thereof, are known as HMG-CoA reductase inhibitors, and used as antihypercholesterolemic and antihyperlipidemic agents in humans. Some of these are available as a base (such as lovastatin, simvastatin, mevastatin and cervistatin) while others are available as a salt to improve their aqueous solubility (for example, pravastatin, atorvastatin and fluvastatin).

Atorvastatin calcium, chemically known as (R—(R*,R*))-2-(4-fluorophenyl)-[beta],[delta]-dihydroxy-5-(1-methylethyl)-3-phenyl-4((phenylamino)carbonyl)-1H-pyrole-1-heptanoic acid hemi calcium salt, is in particular known to inhibit intracellular synthesis of cholesterol, and is considered especially useful in the treatment of hypercholesterolaemia and hyperlipidaemia. In the formula stated, R and R* are moieties that can be any alkaline salt, such as calcium.

Atorvastatin calcium can exist in an amorphous or crystalline form. The amorphous form dissolves more rapidly and is more soluble than the crystalline form, making the amorphous form more commercially desirable for pharmaceutical therapy. Regardless of form (but especially the amorphous form), atorvastatin calcium is highly susceptible to instability brought on by heat, moisture, light and acidic environments (such as gastric content). The instability in acidic condition and poor bioavailability of an HMG-CoA reductase inhibitor like atorvastatin calcium amorphous form requires patients to consume higher dosages with greater frequency to achieve a desired therapeutic result, a problem known to result in poor patient compliance.

Although the amorphous form of atorvastatin calcium is more soluble than its crystalline form, atorvastatin calcium is practically insoluble in water and solubility is pH dependent. That is, at pH of 1.2, the solubility is less than 0.1 mg/ml, while at pH of 8.0, the solubility is increased to 1.0 mg/ml. Higher solubility is desired because higher solubility results in an increase in the bioavailability of atorvastatin calcium. In either form, atorvastatin calcium requires additives to achieve solubility that is sufficient to achieve bioavailability in human subjects. Commonly used solubilizing agents or surfactants, such as polysorbate 80 (Tween 80™), improve solubility by reducing the surface tension action, but react with atorvastatin calcium (especially the amorphous form) to cause destabilization by oxidation. Attempts at stabilizing Atorvastatin calcium from oxidation have been made by using anti-oxidants such as butylated hydroxyl anisole, butylated hydroxyl toluene and sorbic acid. However, these anti-oxidants are not effective in preventing the degradation of atorvastatin calcium, especially its amorphous form, without causing impurities to occur above greater than accepted limits.

Attempts have been made to stabilize atorvastatin calcium exposed to gastric mucosa by adding a buffering or basifying agent (WO 00/35425; WO 94/16603). These solutions are problematic as basifying agents can have a negative impact on gastric mucosa, especially in patients having damaged gastric membranes.

Other attempts have been made to stabilize atorvastatin calcium exposed to gastric mucosa by providing an alkaline medium, as in U.S. Patent Pub. No. 2004247673. This solution tends to produce a relatively impure atorvastatin calcium, containing between 0.30%-0.50% lactone (whereas an acceptable limit should be less than 0.15%).

Still other attempts have been made to stabilize atorvastatin calcium exposed to heat, moisture and light, by packaging pharmaceutical formulations of atorvastatin calcium into packages sealed with inert gases (U.S. Patent Pub. No. 20040077708). This solution is impractical for a number of reasons including that stability (via the inert gas) is lost once the package is opened, and the drug is still otherwise susceptible to rapid degradation in an acidic environment.

Marketable pharmaceutical dosage forms of anti-cholesterolaemic and anti-hyperlipidaemic agents, especially atorvastatin calcium amorphous form, require stability and good bioavailability. The present invention is an anti-cholesterolaemic or anti-hyperlipidaemic drug composition having improved stability and bioavailability, and a method for manufacturing same.

### SUMMARY OF THE INVENTION

One embodiment of the present invention provides a drug composition of improved bioavailability and stability, comprising a pharmaceutical, a complexing agent and a surfactant.

Another embodiment of the present invention provides a drug composition of improved bioavailability and stability, comprising atorvastatin calcium, a cyclodextrin, and a surfactant d-alpha tocopheryl polyethyelene glycol 1000 succinate.

Yet another embodiment of the present invention provides a method for manufacturing the above drug composition comprising the steps of, in a first vessel, dissolving a surfactant in water. A second vessel of water is provided wherein a pharmaceutical and a complexing agent, after dry mixing, are mixed therein to form a slurry. The first vessel contents are then mixed into the second vessel and the complex is stirred and dried to get a dried complex which is meshed through screen to get granules, to which a co-processed composition of starch lactose is added. The composition is blended with a disintegrant and a lubricant and compressed into tablets which are then coated.

### BRIEF DESCRIPTION OF THE DRAWINGS

FIG. 1 is an illustration of the structure of atorvastatin calcium.

FIG. 2 is an illustration of the structure of d-alpha tocopheryl polyethyelene glycol 1000 succinate.

FIG. 3 is an illustration of the structure of beta-cyclodextrin.

FIG. 4 is an illustration of the complexation of a drug inside a hydrophobic cavity of beta-cyclodextrin.

### DETAILED DESCRIPTION

Pharmaceutical compositions containing HMG-CoA reductase inhibitors (such as statins and acceptable statin salts) are stable at basic pH levels. Higher pH levels, preferably greater than 9, yield more stable pharmaceutical grade HMG-CoA reductase inhibitors. Acidic environments like gastric mucosa rapidly destabilize and disintegrate HMG-CoA reductase inhibitors. Rapid destabilization and poor bioavailability requires patients to consume higher dosages with greater frequency, resulting in poor patient compliance and greater frequency of adverse and side effects.

In a preferred embodiment of the present invention, a pharmaceutical (more preferably an HMG-CoA reductase inhibitor and yet more preferably atorvastatin calcium amorphous form) is protected against destabilization in an acidic environment by utilizing cyclodextrin (more preferably beta-cyclodextrin) as an inclusion complexing agent, and has improved solubility and bioavailability by the addition of a surfactant (more preferably d-alpha tocopheryl polyethyelene glycol 1000 succinate).

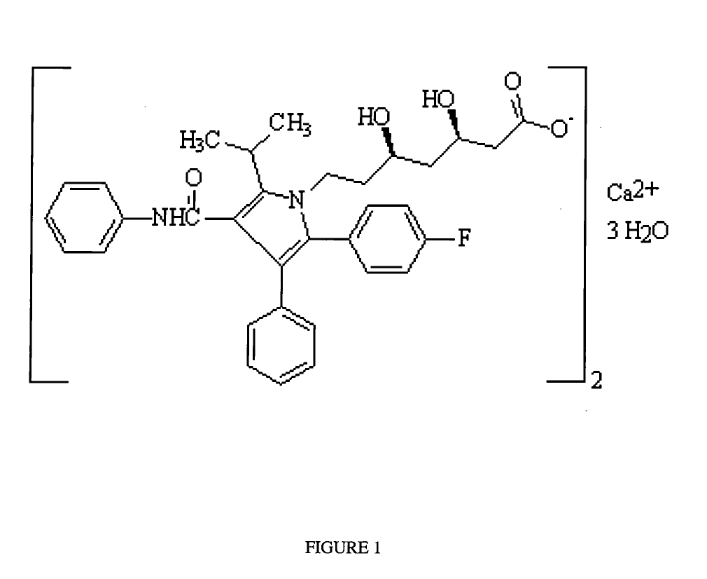

Atorvastatin calcium is a white to off-white powder having an empirical formula of (C₃₃H₃₄FN₂O₅)₂Ca.3H₂O and a molecular weight of 1209.42 (FIG. 1). Although both crystalline and amorphous forms exhibit identical formulary characteristics, they differ in respect of x-ray diffraction patterns and other physiochemical properties.

On human ingestion, atorvastatin calcium lowers plasma cholesterol and lipoprotein levels by inhibiting HMG-CoA reductase and cholesterol synthesis in the liver, and by increasing the number of hepatic low density lipoprotein (LDL) receptors on cell surfaces to enhance uptake and catabolism of LDL. Atorvastatin reduces LDL production and the number of LDL particles. Atorvastatin produces a marked and sustained increase in LDL receptor activity coupled with a beneficial change in the quality of circulating LDL particles. Atorvastatin is rapidly absorbed after oral administration, generally with maximum plasma concentrations occurring within 1 to 2 hours. A constant proportion of atorvastatin is absorbed intact, and the absolute bioavailability is generally 14%. The low systemic availability is attributed to pre-systemic clearance in gastrointestinal mucosa and/or hepatic first-pass metabolism.

In humans, atorvastatin is extensively metabolised to ortho- and para-hydroxylated derivatives. In vitro inhibition of HMG-CoA reductase by ortho- and para-hydroxylated metabolites is equivalent to that of atorvastatin, and approximately 70% of circulating inhibitory activity for HMG-CoA reductase is attributed to active metabolites. In vitro studies suggest atorvastatin is biotransformed by cytochrome P450 3A4, consistent with increased plasma concentrations of atorvastatin in humans following co-administration with erythromycin, a known inhibitor of this isozyme. Atorvastatin is eliminated primarily in bile following hepatic and/or extrahepatic metabolism; however, the drug does not appear to undergo enterohepatic recirculation. Mean plasma elimination half-life of atorvastatin in humans is approximately 14 hours, but the half-life of inhibitory activity for HMG-CoA reductase is 20 to 30 hours due to the contribution of active metabolites. Less than 2% of a dose of atorvastatin is recovered in urine following oral administration.

Drug solubility (or wettability) can be enhanced by a number of methods including 1) chemical modification using a pro-drug concept; 2) physical modification via size reduction and surface modification; 3) pH control using a buffering system; 4) co-solvents; 5) surfactants used as absorption enhancers; and 6) complexation.

A surfactant (generally short for “surface active agent”) is any chemical that, when dissolved in water or another solvent, orients itself at the interface or boundary between two media (for example, solid and liquid) to reduce surface tension, thereby increasing spreading and wetting properties. A surfactant typically has at one end a long non-polar hydrophobic chain, and at another part, a hydrophilic compound.

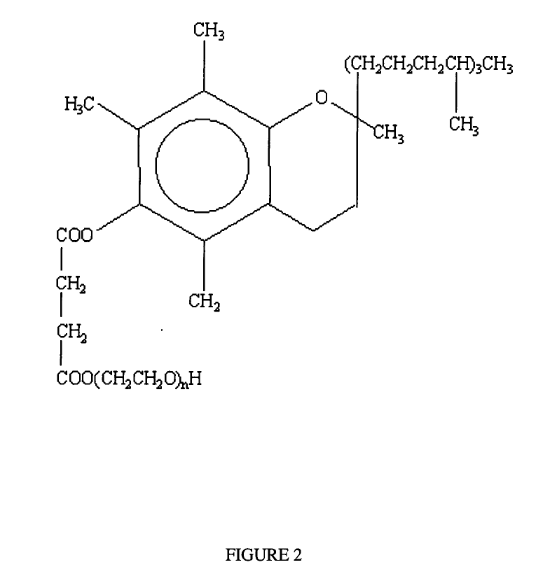

FIG. 2 shows the structure of the surfactant d-alpha-tocopheryl polyethylene glycol-1000 succinate (Vitamin E-TPGS™). Vitamin E-TPGS™ is a polyethylene glycol based surfactant together with a water miscible form of vitamin E derivative (unlike other forms of Vitamin E). It is a waxy solid at room temperature having a melting point of between 37-41° Celsius, and an average molecular weight of approximately 1513. It is water miscible, soluble in polyethylene glycol, has a hydrophilic-lipophilic balance (HLB) value of 13.2, is stable at a pH range of between 4.5-7.5, and has a vitamin E content of 260 mg/g (387 IU/g). Vitamin E is known to have beneficial anti-oxidant properties.

Vitamin E-TPGS™ is formed by esterifying d-alpha-tocopheryl acid succinate with polyethylene glycol 1000. It may be incorporated into water-based oral vitamin supplements, providing a bioavailable source of vitamin E for individuals having difficulties absorbing fat-soluble vitamin E forms. Because of its chemical functionality, it can emulsify fat-soluble actives and may enhance their bioavailability.

Vitamin E-TPGS™ forms its own micelles and can be absorbed by malabsorbers. In water, it coils itself with the polyethylene glycol part that is miscible in water on the outside and the non-miscible part on the inside. This allows it to traverse easily in water and carry inside it, fat-soluble or hydrophobic compounds.

The surfactant properties of Vitamin E-TPGS™ improve the solubility of atorvastatin calcium without reacting with atorvastatin calcium to cause destabilization by oxidation. The vitamin E properties of Vitamin E-TPGS™ stabilizes atorvastatin calcium from degredation by oxidation without causing impurities to occur in greater than accepted amounts, while also enhancing the bioavailability of atorvastatin calcium.

Complexation, the reversible association of a substrate and ligand to form a new species, is one way to favorably enhance the physicochemical properties of pharmaceutical compounds. Cyclodextrins are examples of compounds that form inclusion complexes. These complexes are formed when a “guest” molecule is partially or fully included inside a “host” molecule with no covalent bonding. When inclusion complexes are formed, the physicochemical parameters of the guest molecule are disguised or altered, and improvements in the molecule's solubility, stability, taste, safety and bioavailability are commonly seen.

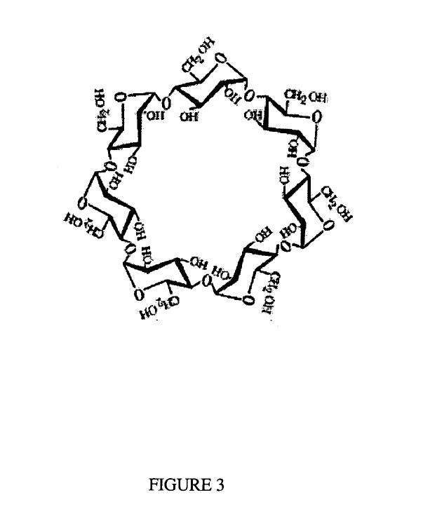

Cyclodextrins are cyclic oligosaccharides containing 6, 7, or 8 glucopyranose units, referred to as alpha, beta or gamma cyclodextrin, respectively. Each glucose unit contains two secondary alcohols at C-2 and C-3, and a primary alcohol at the C-6 position, providing 18-24 sites for chemical modification and derivatization. The chemical structure of beta-cyclodextrin is shown in FIG. 3.

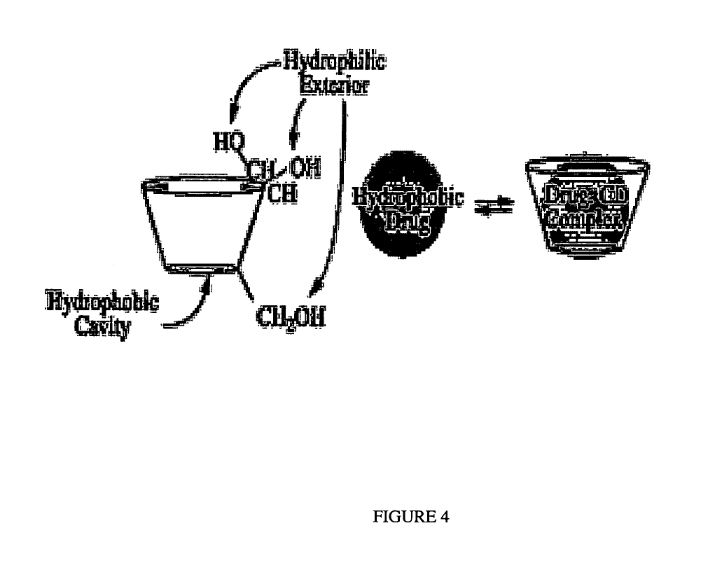

FIG. 4 shows cyclodextrin defining a hydrophobic cavity relative to an aqueous environment. Sequestration of hydrophobic drugs inside the cyclodextrin cavity can improve a drug's solubility and stability in water, the rate and extent of dissolution of the drug:cyclodextrin complex, and the bioavailability of the drug when dissolution and solubility are limiting the delivery. These cyclodextrin properties enable insoluble drug formulations that are typically difficult to formulate and deliver with more traditional excipients.

A cyclodextrin inclusion complex is resistant to hydrolysis in the acidic environment of the stomach, thus maintaining an active drug ingredient as a guest within the inclusion complex following oral administration. This permits the active drug ingredient to pass through the stomach and resist degradation and destabilization in the acidic environment of the stomach. However, the inclusion complex is not resistant to digestion by enzymes present in the liver and in the intestinal region, thus causing its breakdown and the release of the active drug ingredient for absorption. In some cases, the drug is released from the inclusion complex upon dilution with contributions from competitive displacement with endogenous lipophiles binding to plasma and tissue components where drug uptake into tissues is not available to the complex and the beta-cyclodextrin is rapidly eliminated.

The present invention can be manufactured using the following steps, using for example atorvastatin calcium amorphous form as the pharmaceutical of choice, beta cyclodextrin as the complexation agent of choice, and d-alpha-tocopheryl polyethylene glycol-1000 succinate as the surfactant of choice.

Two hundred milliliters of purified water (or any other suitable vehicle) is measured into a suitable vessel, and heated to approximately sixty degrees Celsius, to which forty grams of Vitamin E TPGS™ is stirred in, forming a solution, which is then allowed to cool to room temperature.

Five hundred milliliters of purified water (or any other suitable vehicle) is measured into a suitable second vessel and fitted with a mechanical stirrer capable of rotation at speeds of about one thousand to two thousand revolutions per minute. Separately, Atorvastatin calcium amorphous form (216.878 grams) and beta cyclodextrin (1.380 kilograms) are dry mixed together in a polyethene bag, and then slowly added in small lots to the second vessel, resulting in a slurry that is continuously stirred. The formation of lumps should be avoided.

Once all of the atorvastatin calcium and beta cyclodextrin has been added and a smooth slurry has been formed, the first vessel contents are then stirred into the second vessel containing the slurry, and stirring is continued for another period of approximately three hours, resulting in a complexed mass. The resulting complexed mass is dried in tray dryer and then screened through a mesh to form dry granules.

A starch-lactose mixture (e.g. Starlac™) (1.280 kilograms) or any other suitable dilutent is added to the granules, together with a suitable disintegrant and lubricating agent, such as Magnesium stearate. The pH of the granules at this stage should be approximately 6.9.

A suitable lubricant (e.g. 30.00 g of magnesium stearate) and disintegrant (e.g. Calcium Carbophil CA-1™) are added to the granules, which are subsequently stored into a clean double polythene bag lined drum for storage.

The granules can subsequently be compressed into tablets. The recipe above can yield almost five thousand tablets of the following specification: average weight—600 mg/tablet; thickness—5.4±02 mm; hardness—10-11 kp; disintegration time—no more than 10 minutes; dissolution—no less than 85% in 45 minutes.

Tablets should preferably be coated, to facilitate human ingestion. A Coating solution using a suitable protective coating like Kollicoat IR™ can be applied after dispersion and stirring in water. In this case, the final solid content should be twenty percent, and should be stirred for forty-five minutes before starting the coating process. Tablets can subsequently be packaged for commercial sale.

While the subject invention has been described and illustrated with reference to certain particular embodiments thereof, those skilled in the art will appreciate that various adaptations, changes, modifications, substitutions, deletions or additions of procedures and protocols may be made without departing from the scope of the invention.

What is claimed is:

1. A drug comprising:
a) a pharmaceutical;
b) a complexing agent; and
c) a surfactant.

2. The drug as claimed in claim 1 wherein the pharmaceutical is one selected from a group consisting of an anti-hypercholesterolaemic agent and an anti-hyperlipidaemic agent.

3. The drug as claimed in claim 1 wherein the pharmaceutical is one selected from a group of a 3-hydroxy-3-methylglutaryl coenzyme A reductase inhibitor, an atorvastatin, atorvastatin calcium and atorvastatin calcium amorphous form.

4. The drug as claimed in claim 1 wherein the complexing agent is a cyclodextrin.

5. The drug as claimed in claim 1 wherein the complexing agent is one selected from a group consisting of alpha-cyclodextrin, beta-cyclodextrin and gamma-cyclodextrin.

6. The drug as claimed in claim 1 wherein the surfactant is a polyethylene glycol-based surfactant containing vitamin E.

7. The drug as claimed in claim 1 wherein the surfactant is d-alpha tocopheryl polyethylene glycol 1000 succinate.

8. The drug as claimed in claim 1 wherein:
a) the pharmaceutical is atorvastatin calcium;
b) the complexing agent is a cyclodextrin; and
c) the surfactant is d-alpha tocopheryl polyethyelene glycol 1000 succinate.

9. The drug as claimed in claim 1 further comprising d) a lubricant and e) a disintegrant.

10. The drug as claimed in claim 8 further comprising d) a lubricant and e) a disintegrant.

11. A method for manufacturing a drug comprising:
a) dissolving a surfactant in water to form a slurry;
b) mixing a pharmaceutical and complexing agent to form a mixture;
c) adding the mixture to the slurry to form a complex mass;
d) drying the complex mass; and
e) meshing the dried complex mass to form granules.

12. The method as claimed in claim 11 wherein the pharmaceutical is one selected from a group consisting of an anti-hypercholesterolaemic agent and an anti-hyperlipidaemic agent.

13. The method as claimed in claim 11 wherein the pharmaceutical is one selected from a group consisting of a 3-hydroxy-3-methylglutaryl coenzyme A reductase inhibitor, atorvastatin, atorvastatin calcium and atorvastatin calcium amorphous form.

14. The method as claimed in claim 11 wherein the complexing agent is a cyclodextrin.

15. The method as claimed in claim 11 wherein the complexing agent is one selected from a group consisting of alpha-cyclodextrin, beta-cyclodextrin and gamma-cyclodextrin.

16. The method as claimed in claim 11 wherein the surfactant is a polyethylene glycol-based surfactant containing vitamin E.

17. The method as claimed in claim 11 wherein the surfactant is d-alpha tocopheryl polyethylene glycol 1000 succinate.

18. The method as claimed in claim 11 further comprising i) adding a dilutent to the granules.

19. The method as claimed in claim 18 further comprising j) applying a disintegrant to the granules.

20. The method as claimed in claim 19 further comprising k) applying a lubricant to the granules.

21. The method as claimed in claim 20 further comprising i) compressing the granules into tablets.

22. The method as claimed in claim 21 further comprising j) applying a coating to the tablets.
